(19) United States
(12) Patent Application Publication
(10) Pub. No.: US 2021/0128461 A1
(43) Pub. Date: May 6, 2021

(54) Method of Delivering Amphiphilic Bioactive Substances Targeting the Respiratory Tract

(71) Applicant: Wise Ally Holdings Ltd, Hong Kong (CN)

(72) Inventors: Erik Chun Hay KO, Hong Kong (CN); Connie Sau Kuen KWOK, Hong Kong (CN)

(21) Appl. No.: 15/733,624

(22) PCT Filed: Mar. 25, 2019
(86) PCT No.: PCT/CN2019/079445

§ 371 Date: Sep. 18, 2020

Related U.S. Application Data
(60) Provisional application No. 62/647,843, filed on Mar. 26, 2018.

(51) Int. Cl. A61K 9/00; A61K 31/724; A61K 36/61; A61K 36/78; A61K 47/26; A61K 47/10; A61M 11/04

(52) U.S. Cl. CPC A61K 9/0073; A61K 31/724; A61K 36/61; A61M 11/042; A61K 47/26; A61K 47/10; A61K 36/78

(57) ABSTRACT

The present invention provides a water-based formulation, comprising hydroxypropyl-β-cyclodextrin, an essential oil selected from a group consisting of Eucalyptus Globubus oil and Houttuynia cordata oil, for targeting the respiratory system, in particular for the upper respiratory system. The formulation is delivered as vapors through an inhaler with a controlled heater.

### FIELD OF INVENTION

This invention relates to a composition for treating inflammatory disease of respiratory tract, in particular, for treating inflammatory disease of upper respiratory tract.

### BACKGROUND OF INVENTION

Asthma and chronic obstructive pulmonary disease (COPD) are obstructive airway diseases affecting millions of people globally. Bronchodilators and anti-inflammatory drugs are common medications for relieving mild to moderate symptoms associated with the diseases. However, the drugs are effective in patients in short-term only and are associated with a number of systemic side effects. Meanwhile, medications delivered by commercially available inhaler devices are only limited to water-soluble drugs, while water-insoluble drugs cannot be effectively delivered to relieve respiratory discomfort. Furthermore, drug delivery by the inhalers does not synchronize with inhalation and may cause over or under dose during administration. There is a need to develop a new and effective drug delivery system for better management of respiratory diseases.

### SUMMARY OF INVENTION

The present invention relates to a composition that vaporizes into vapors with a droplet size of 1-10μm when the composition is at a temperature of 65-75° C.

One example embodiment is a composition which includes hydroxypropyl-β-cyclodextrin (HP-CD), an essential oil, water, optionally a surfactant and optionally a stabilizer. The essential oil is selected from a group consisting of Eucalyptus Globulus oil and Houttuynia cordata oil. The surfactant is polysorbate. The stabilizer is polyhydroxy alcohol.

Other example embodiments are discussed herein.

### BRIEF DESCRIPTION OF FIGURES

FIG. 1 shows vaporized amount of α-pinene, limonene and eucalyptol from formulation 1 (a), 2 (b) and 3 (c) heated at 65° C., 80° C. and 90° C. (n=3).

FIG. 2 shows relationship between maximum Eucalyptus Globulus oil loading and polysorbate 20-to-HP-CD (T20/HP-CD). Curve fitting by OriginLab.

FIG. 3 shows typical size distribution of vapors from formulation 5 at 65° C.

FIG. 4 shows weight distribution of deposited vaporized content from formulation 5 in different NGI stages (n=3) at 37 L/min pump flow rate. The cut-off size of stages 1-7 were 10.464, 5.735, 3.591, 2.083, 1.215, 0.735 and 0.470 μm, respectively.

FIG. 5 shows vaporization of formulation 5 during 30-minute heating. (a): α-pinene; (b): limonene; (c): eucalyptol.

FIG. 6 shows amount of APIs deposited in organs at different inhalation times in in vivo vapor droplet deposition study (n=4-5; outliners were excluded for mean calculation).

FIG. 7 shows distribution of API in the upper respiratory system (URS) and the whole respiratory system (RS) at different inhalation times in in vivo vapor deposition study. (n=4-5; outliners were excluded for mean calculation).

### DETAILED DESCRIPTION

Example embodiments relate to a composition/formulation that includes hydroxypropyl-β-cyclodextrin (HP-CD), an essential oil, water, optionally a surfactant and optionally a stabilizer. The essential oil is Eucalyptus Globulus oil or Houttuynia cordata oil. The surfactant is polysorbate. The stabilizer is polyhydroxy alcohol. When the composition/formulation is at a temperature of 65-75° C., it can be delivered in the form of vapors with a droplet size of 1-10 μm to the respiratory tract of a patient.

Eucalyptus oil suppresses arachidonic acid metabolism and cytokine production involved in inflammation, and possesses some medical functions such as expectorant, antitussive, nasal decongestant, analgesic, antimicrobial, antioxidant, anti-inflammatory and antispasmodic. Eucalyptus oil provides an alternative approach for relieving asthma and COPD symptoms. Functional compounds (or active pharmaceutical ingredients) in the Eucalyptus oil including eucalyptol, pinene and limonene have anti-inflammatory, bronichodilating and immunostimulant properties. Inhalation of the active ingredients in Eucalyptus oil is beneficial to asthma and COPD patients.

Houttuynia cordata oil has a function of enhancing immunity system, anti-pathogenic bacteria, anti-allergy, and anti-inflammation. Houttuynia cordata oil has effect in treating asthma and COPD patients. Houttuynia cordata oil can treat or alleviate upper respiratory discomfort.

In order to deliver the essential oil into the respiratory tract via inhalation to treat diseases such as asthma and COPD, the essential oil should be delivered in vapor form, which can be achieved by boiling. However, the boiling point of the essential oil is very high. For example, the boiling point of Eucalyptus oil is in the range of 176-177° C. under atmospheric pressure which is difficult to achieve and would result in pyrolysis and formation of toxicant and hazardous substances. Example embodiments solves the problems by lowering the boiling point of the essential oil to a temperature below 75° C. by formulating Eucalyptus Globulus oil or Houttuynia cordata oil in an aqueous-based solvent, and generating sufficient amount of vapors of the formulation during heating at this lower temperature (i.e. lower than 75° C.). Given that the essential oil is not miscible with water, amphiphilic excipients including cyclodextrin and optionally polysorbate/polyhydroxy alcohol are employed to solubilize the Eucalyptus Globulus oil/Houttuynia cordata oil in the formulation.

Cyclodextrins are cyclic oligosaccharides derived from starch. The inner surface of the toroidal structure is hydrophobic, while the outer surface is hydrophilic. The hydrophobic captivity can trap water-insoluble molecules, while the hydrophilic surface gives rise to the water solubility of the essential oil-cyclodextrin complex.

In one example embodiment, cyclodextrins include α-cyclodextrin, β-cyclodextrin, and γ-cyclodextrin. In one example, cyclodextrins includes hydroxypropyl-β-cyclodextrin (HP-CD). HP-CD encapsulates Eucalyptus Globulus oil or Houttuynia cordata oil in inner surface and its outer surface is hydrophilic so that HP-CD improves the solubility of Eucalyptus Globulus oil or Houttuynia cordata oil in water, and reduce oil degradation and vaporization during storage. When HP-CD encapsulates Eucalyptus Globulus oil or Houttuynia cordata oil in the inner surface, the solution including HP-CD and the essential oil and water can be a clear solution.

In one example embodiment, polysorbate (also known as TWEEN®) solubilizes the essential oil e.g. Eucalyptus Globulus oil in aqueous formulation. Different chain lengths of the molecules possess different hydrophile-lipophile balance (HLB) values. HLB values of polysorbate 20, 40 and 60 are 16.7, 15.6 and 14.9, respectively. In one example embodiment, the composition/formulation includes polysorbate 20. In one example embodiment, polyhydroxy alcohol is propylene glycol.

In one example embodiment, the composition/formulation includes 1.6%-28% HP-CD, 3%-25% polysorbate 20, 0.8%-7% Eucalyptus Globulus oil, and 41%-94% water in weight % of the composition. In one example embodiment, the composition consists essentially of 1.65%-27.2% HP-CD, 3.24%-24.4% polysorbate 20, 0.825%-6.8% Eucalyptus Globulus oil, and 41.52%-93.57% water in weight % of the composition.

In one example embodiment, the composition consists essentially of about 3.6% HP-CD, about 3.24% polysorbate 20, about 0.825% Eucalyptus Globulus oil, and about 92.26% water in weight% of the composition. In one example embodiment, the composition consists essentially of about 1.65% HP-CD, about 3.96% polysorbate 20, about 0.825% Eucalyptus Globulus oil, and about 93.57% water in weight % of the composition.

In one example embodiment, the composition consists essentially of about 27.2% HP-CD, about 24.4% polysorbate 20, about 6.8% Eucalyptus Globulus oil, and about 41.52% water in weight % of the composition. In one example embodiment, the composition consists essentially of about 9.06% HP-CD, about 8.13% polysorbate 20, about 2.27% Eucalyptus Globulus oil, and about 80.54% water in weight % of the composition.

In one example embodiment, the composition consists essentially of about 14.8% HP-CD, about 0.68% Eucalyptus Globulus oil, about 79.52% water, and about 5% propylene glycol. In one example embodiment, the composition consists essentially of about 11% HP-CD, about 0.18% Houttuynia cordata oil and about 88.82% water.

In one example embodiment, the composition includes 0.3%-0.7% Houttuynia cordata oil, 5%-11% polysorbate 20, 5%-11% HP-CD, and 77%-89% water in weight % of the composition.

In one example embodiment, the composition consists essentially of 0.35%-0.7% Houttuynia cordata oil, 5.5%-10.9% polysorbate 20, 5.5%-10.9% HP-CD, and 77.58%-88.65% water in weight % of the composition. In one example embodiment, the composition consists essentially of about 0.35% Houttuynia cordata oil, about 5.5% polysorbate 20, about 5.5% HP-CD, about 88.65% water in weight% of the composition.

In one example embodiment, the formulation/composition is in the form of a vapor, and the droplet size of the vapor is 1-10 μm. In one example embodiment, the vapor is at a temperature of between about 65 and 75° C. The composition/formulation is formed in an inhaler. In one example embodiment, the composition/formulation is contained in a nebulizer.

In one example embodiment, the formulation/composition vaporizes into droplet/particle sizes of 1-10 μm when the formulation/composition is at a temperature of 65-75° C. The droplet/particle sizes are inhalable sizes, which enhances the delivery efficiency of the droplets/particles to the respiratory tract of the user. The droplets/particles with a size of 1-10 μm can effectively be deposited in the respiratory tract of the user and thereby can treat inflammatory diseases associated with respiratory tract.

In one example embodiment, more than 50% of the droplets/particles generated from the formulation/composition have a size of 4-10 μm which makes the droplets/particles effectively deposit in the upper respiratory tract of the user, which is used to treat diseases associated with upper respiratory tract. In one example embodiment, the diseases associated with upper respiratory tract includes asthma and COPD, etc.

In one example embodiment, the temperature at which the composition/formulation is below 70° C. In one example embodiment, the temperature at which the composition/formulation is 65° C. In one example embodiment, vapors from the formulation at 65° C. have a droplet size of 7.20±0.36 μm. The mass median aerodynamic diameter of the vapors was measured to be 8.48±0.26 μm at 37 L/min pump flow rate, while over 70% of the deposits were larger than 4 μm.

In one example embodiment, the HP-CD fully encapsulates the essential oil in hydrophobic cavities of the HP-CD so that the essential oil is solubilized in the water.

Example embodiments provide an inhaler where the formulation/composition described herein is contained or stored. The formulation/composition is delivered as vapors through the inhaler, and the vapors have droplets/particles with a size of 1-10 μm, in particular 4-10 μm when the formulation/composition is at a temperature of 65-75° C. In one example embodiment, the inhaler is a nebulizer.

Existing nebulizers usually generate vapor droplets through vibration of the drug solution therein. Nevertheless, extra accessories such as ultrasonicator or compressor are required to generate aerosols, the size of the overall device is bulky and limited to hospital or household use only. Further, nebulizers are only compatible with hydrophilic drugs as hydrophobic drugs cannot be effectively dispersed in aqueous drug solution and released through vibration. Example embodiments solve the problems by formulating the composition described herein which makes Eucalyptus Globulus oil/Houttuynia cordata oil soluble in water and can be delivered as vapors below 75° C., or below 70° C., or preferably at 65° C.

MDI can carry powder or solution form of drugs, which are delivered through compressed propellant to form aerosols. However, the device can only be inhaled through the mouth. As a result, the majority of drugs will be trapped in the mouth cavity and cannot be delivered to the target site of the respiratory system. Example embodiments solve the problems by providing an inhaler which synchronizes with inhalation and prevents over or under dose during administration.

One example embodiment provides an inhaler including a pod and a heater. The pod includes the composition/formulation discussed herein. The heater is configured to heat the composition/formulation to 65-75° C.

In one example embodiment, the heater is configured to heat the composition/formulation to below 70° C. In one example embodiment, the heater is configured to heat the composition/formulation to 65° C.

Example embodiments provide a method of treating inflammation disease of the respiratory tract in a subject in need thereof. The method includes administering the composition/formulation described herein to the subject. The composition/formulation is contained in an inhaler. In one example embodiment, the inflammation disease can be asthma or chronic obstructive pulmonary disease.

In one example embodiment, when the formulation/composition is heated to a temperature of 65-75° C., below 70° C., or at 65° C., it vaporizes into droplets with a size of 1-10 μm. The droplets with 1-10 μm including active pharmaceutical ingredients of the essential oil can deposit into the respiratory tract of the subject and thereby treat the disease of the respiratory tract.

In one example embodiment, when the formulation/composition is heated to a temperature of 65-75° C., below 70° C., or at 65° C., it vaporizes into droplets with a size of 4-10 μm. The droplets with 4-10 μm including active pharmaceutical ingredients of the essential oil can deposit into the upper respiratory tract of the subject and thereby treat the disease of the upper respiratory tract.

In one example embodiment, vapors of the composition are inhaled by the subject for 1-10 minutes to treat the inflammation disease of the upper respiratory tract. In one example embodiment, vapors of the composition are inhaled by the subject for 1-5 minutes to treat the inflammation disease of the upper respiratory tract.

In one example embodiment, more than 50% of APIs are deposited into the upper respiratory tract from 1-5 minute inhalation time. The subject inhales the vapors of the composition/formulation for 1-5 minutes to treat the inflammation disease of upper respiratory tract. In one example embodiment, the subject inhales the vapors of the composition/formulation for 10-15 minutes to treat the inflammation disease of whole respiratory tract. In some embodiments, API stands for active pharmaceutic ingredient. In some embodiments, it refers to the one or more functional compounds in the essential oil. For example, in Eucalyptus Globulus oil, the APIs are a-pinene, limonene, and eucalyptol.

Example embodiments provide a method of making the composition/formulation described herein. HP-CD is mixed into water to obtain a HP-CD solution. The essential oil and optionally the surfactant/optionally the stabilizer is added to the HP-CD solution at room temperature to obtain a mixture. In some embodiments, the mixture is vortexed and then degassed to obtain the formulation/composition. In some embodiments, the surfactant is polysorbate. In some embodiments, the stabilizer is polyhydroxy alcohol.

In one example embodiment, 1.6%-28% (w/w) HP-CD is mixed into 41%-94% (w/w) water to obtain the HP-CD solution. 0.8%-7% (w/w) Eucalyptus Globulus oil and 3%-25% (w/w) polysorbate 20 are added to the HP-CD solution with constant stirring at room temperature to obtain the mixture. The mixture is vortexed for 1 minute and degassed for 30 seconds to obtain the formulation/composition.

In one example embodiment, 1.65%-27.2% (w/w) HP-CD is mixed into 41.52%-93.57% (w/w) water to obtain the HP-CD solution. 0.825%-6.8% (w/w) Eucalyptus Globulus oil and 3.24%-24.4% (w/w) polysorbate 20 are added to the HP-CD solution with constant stirring at room temperature to obtain the mixture. The mixture is vortexed for 1 minute and degassed for 30 seconds to obtain the formulation/composition.

As used herein and in the claims, the term “about” when used before a numerical designation, e.g., temperature, time, amount, percentage, and concentration, including a range, indicates approximations which may vary by ±10%, ±5% or ±1%.

As used herein and in the claims, the term “essential oil” refers to a concentrated hydrophobic liquid comprising one or more API(s).

As used herein and in the claims, the term “subject” is used herein in its broadest sense. In certain embodiments, a subject can be an animal, particularly an animal selected from a mammalian species including rat, rabbit, bovine, ovine, porcine, canine, feline, murine, equine, and primate, particularly human.

### EXAMPLES

### Example 1—Instruments

Gas Chromatography-Flame Ionization Detector (GC-FID)

GC-FID analysis was performed on an Agilent 7890A instrument equipped with a flame ionization detector and a DB-5MS column (30 m×0.32 mm×0.25 μm) capillary column. Nitrogen was used as the carrier gas at a flow rate of 1 mL/min and the volume injected was 1 μL in splitless mode. The oven temperature was initiated at 45° C. for 3 min, then increased to 85° C. at 8° C./min and held for 1 minute. The column temperature was then increased to 100° C. at 5° C./min, followed by 120° C. at 4° C./min. The temperature was ramped to 280° C. at 40° C./min and held for 5 minutes. Injection port and detector temperatures were maintained at 200° C. and 280° C., respectively. Analyte content was analyzed from calibration curves constructed from peak areas at different retention times on the spectra. Retention time (min) of reference standards: 14.02 (α-pinene), 17.00 (limonene), 17.14 (eucalyptol).

Gas Chromatography-Mass Spectrometer (GC-MS)

GC-MS analysis was performed on an Agilent 7890B instrument equipped with an Agilent 5977B mass spectrometry detector and a DB-5MS column (30 m×0.25 mm×0.25 μm) capillary column. Helium was used as the carrier gas at a flow rate of 1.2 mL/min and the volume injected was 1 μL in splitless mode. The oven temperature was initiated at 45° C. for 3 min, then increased to 85° C. at 8° C./min and held for 1 minute. The column temperature was then increased to 100° C. at 5° C./min, followed by 120° C. at 4° C./min. The temperature was ramped to 280° C. at 40° C./min and held for 5 minutes. Post run temperature was maintained at 45° C. for 4 minutes. Injection port and auxiliary heater temperatures were maintained at 300° C. and 280° C., respectively. Analytes were detected by the mass spectrometer after 5-minute solvent delay time. Analyte content was analyzed from calibration curves constructed from peak areas at different retention times and mass-to-charge ratios (m/z) on the spectra using Selected Ion Monitoring (SIM) mode. Mass-to-charge (m/z) and quantifier of reference standards at SIM time segment=5 min: α-pinene [77, 93 (quantifier)]; limonene [93, 121 (quantifier)]; eucalyptol [111 (quantifier), 154].

Laser Diffraction System

The droplet size of vapors was characterized by a laser diffraction system (Malvern). The instrument was operated in an open configuration and equipped with a 5 mW helium-neon laser to scatter droplets at 633 nm for measurement. Particle size distribution of the droplets was obtained from the laser diffraction software. Number distribution of scattered particles was reported in Dn(10), Dn(50) and Dn(90), which corresponded to the diameter of particles separating the lower 10%, 50% and 90% of a distribution.

Next Generation Impactor (NGI)

Aerodynamic droplet size of vapors were characterized by NGI (Colpley Scientific). The outlet of the instrument was connected to a HCPS high capacity vacuum pump (Copley Scientific) to generate air flow passing through the impactor. The NGI system was operated at 37 L/min. The cup-off diameters of the collection cups at stages 1-7 and micro-orifice collector (M° C.) were 10.464 μm, 5.735 μm, 3.591 μm, 2.083 μm, 1.215 μm, 0.735 μm and 0.470 μm correspondingly. Mass median aerodynamic diameter (MMAD) of the deposited vapors was determined by Copley Inhaler Testing Data Analysis Software (CITDAS) (Coley Scientific).

### Example 2

Preparation of Formulation 1:

HP-CD (1.0 g) was first dissolved in water (10 g) and Eucalyptus Globulus oil (0.1 mL) was added to the HP-CD solution with constant stirring at room temperature. The mixture was vortexed for 1 minute and allowed to stir for 15 minutes to give a clear formulation.

Preparation of Formulations 2-5:

HP-CD was dissolved in water and a mixture of Eucalyptus Globulus oil and polysorbate 20 (T20) was added to the HP-CD solution with constant stirring at room temperature. The mixture was vortexed for 1 minute, followed by degassing for 30 seconds to give a pale yellow formulation. Compositions of the formulations are summarized in Table 1 below.

TABLE 1
Preparation of formulations 2-5

| | | | Eucalyptus | | Ratio |
|---|---|---|---|---|---|
| Formulation # | HP-CD | T20 | Globulus oil | Water | (T20/HP-CD) |
| 2 | 0.360 g | 0.324 g | 0.0825 g | 9.226 g | 0.9 |
| | (3.60% w/w) | (3.24% w/w) | (0.09 mL; | (92.26 w/w) | |
| | | | 0.825% w/w) | | |
| 3 | 0.165 g | 0.396 g | 0.0825 g | 9.357 g | 2.4 |
| | (1.65% w/w) | (3.96% w/w) | (0.09 mL; | (93.57 w/w) | |
| | | | 0.825% w/w) | | |
| 4 | 2.72 g | 2.44 g | 0.68 g | 4.152 g | 0.9 |
| | (27.2% w/w) | (24.4% w/w) | (0.743 mL; | (41.52 w/w) | |
| | | | 6.8% w/w) | | |
| 5 | 0.906 g | 0.813 g | 0.227 g | 8.054 g | 0.9 |
| | (9.06% w/w) | (8.13% w/w) | (0.248 mL; | (80.54 w/w) | |
| | | | 2.27% w/w) | | |

Preparation of Formulation 6:

HP-CD (16.28g) was dissolved in distilled water (87.47 g) and stirred for 15 minutes at 600 rpm. The solution was filtered by 0.2 μm PES membrane. Eucalyptus Globulus oil (0.680 g) was added to the filtrate (94.32 g) and stirred for 18 hours at 600 rpm to give a clear solution. Propylene glycol (PG) (5 g) was added to the solution, followed by and stirring at 600 rpm for 10 minutes to give a clear solution. The preparation of formulation 6 is summarized in table 2.

TABLE 2
Preparation of formulation 6

| | | | Eucalyptus | | Oil in |
|---|---|---|---|---|---|
| Formulation # | HP-CD | PG | Globulus oil | Water | formulation |
| 6 | 16.28 g | 5 g | 0.680 g | 87.47 g | 6.8 mg/g |
| | (14.80% w/w) | (5.00% w/w) | (0.68% w/w) | (79.52 w/w) | |

Preparation of Formulation 7:

HP-CD (12.10 g) was dissolved in distilled water (97.70 g) and stirred for 15 minutes at 600 rpm. The solution was then filtered by 0.2 μm PES membrane. Houttuynia cordata oil (0.180 g) was added to the filtrate (99.82 g) and stirred for 18 hours at 600 rpm to give a clear solution. The preparation of formulation 7 is summarized in table 3.

TABLE 3
shows preparation of formulations 7

| | | Houttuynia | | Oil in |
|---|---|---|---|---|
| Formulation # | HP-CD | cordata oil | Water | formulation |
| 7 | 12.10 g | 0.180 g | 97.70 g | 1.8 mg/g |
| | (11% w/w) | (0.18% w/w) | (88.82 w/w) | |

Preparation of Formulation 8:

Houttuynia cordata oil (0.035 g) is mixed with polysorbate 20 (0.55 g) by stirring for 5 minutes. The solution is labeled as “Solution A”. In a separate vial, HP-CD (0.55 g) is dissolved in aromatic water of Houttuynia cordata (8.865 g) by vortexing for 1 minute. The solution is labeled as “Solution B”. Solution A is then combined with Solution B, followed by vortexting for 1 minute and degassing for 30 seconds to give a clear solution.

### Example 3—Analysis of Vaporization Amount at Different Temperatures by GC-FID

Active pharmaceutic ingredient (API) content in vapors from the heated formulations was analyzed by GC-FID. Inhaler device was first pre-heated to either 65° C., 80° C. or 90° C. The exhaust of the inhaler device was connected to a pump (flow rate=1.2 L/min), the outlet of which was connected to the valve of an air sampling bag. About 1 mL of the formulation was then fed into the pod of the inhaler and heated to the designated temperature for 1 minute. The pump was then switched on for 40 seconds to direct the generated vapors into the air sampling bag. After sealing the air sampling bag, 5 mL of n-hexane was injected into the bag to dissolve the collected vapors. The rinsed hexane solution was then collected for GC-FID analysis of α-pinene, limonene and eucalyptol content.

### Example 4—Determining the Droplet Size of Vapors from the Formulation/Composition

Droplet size of vapors was measured by a laser diffraction system. A glass petri dish one-third filled with formulation was heated to 65° C. between the transmitter and receiver of the instrument with constant stirring. Signals from the vapors were collected for at least 5 minutes. Results in 1-minute intervals were averaged. Triplicate measurements were performed for all samples.

### Example 5—In Vitro Evaluation of the Aerodynamic Droplet Size of Vapors from the Formulation/Composition

Aerodynamic droplet size of vapors was measured by an in vitro impactor model. A 100-mL round-bottom flask containing 20 mL of the formulation was connected to the NGI system. The formulation was heated to 65° C. and the NGI system was pumped at a flow rate of 37 L/min for 10 minutes. The temperature of the formulation was maintained at 65° C. throughout the pumping process. The deposited content in the collection stages 1-7 and M° C. was weighed immediately after pumping to avoid evaporation. The average Mass median aerodynamic diameter (MMAD) of the vapors was obtained by analyzing the weight of the residues with Copley Inhaler Testing Data Analysis Software (CITDAS). Triplicate measurements were performed for all samples.

### Example 6—Screening for Harmful Chemicals in Formulation Generated During Heating

The formulation/composition was screened for harmful organic chemicals in the vapors through outsourcing. The formulation was refluxed at 100° C. for an hour and sent to accredited testing and certification laboratory for analysis. The screening was performed in accordance with a modified USP Chapter <467> protocol, i.e. headspace GC-MS was used instead of headspace GC-FID detection system.

### Example 7—Evaluation of API Content in Vapors from Formulation during 30-minute Heating

API content in vapors released from the formulation during 30-minute heating was analyzed by GC-MS. Inhaler device was first pre-heated to 65° C. The exhaust of the inhaler device was connected to a pump (flow rate=6.0 L/min). About 1 mL of the formulation was then fed into the pod of the inhaler and heated to the designated temperature for 1 minute. The pump was then switched on for 30 minutes continuously. At 1, 5, 10, 15, 20, 25 and 30-minute time point, about 0.4-0.5 L of the vapors were collected by connecting an air sampling bag to the outlet of the pump. After sealing the air sampling bag, 5 mL of n-hexane was injected into the bag to dissolve the collected vapors. The rinsed hexane solution was then collected for GC-MS analysis.

### Example 8—Results

1. Preparation of Formulations:

A series of water-based Eucalyptus Globulus oil and Houttuynia cordata oil formulations were prepared. Solubilization of the essential oil in water was achieved through the addition of HP-CD and/or polysorbate 20 and/or propylene glycol. This required mixing the ingredients at room temperature until the solution became clear. Formulation with HP-CD in the absence of polysorbate 20 required longer preparation time. The extra 15-minute mixing step ensured all the essential oil was encapsulated within the hydrophobic cavity of HP-CD, which was indicated by the transformation of the solution from turbid to clear throughout the mixing.

Formulation 1 showed that HP-CD alone had the capacity to solubilize the same amount of essential oil in water without the use of surfactant. Nevertheless, a relatively high production cost would be involved for large-scaled production of the formulation due to the use of costly HP-CD with high molar substitution. Polysorbate with a HLB of 16.7, i.e. polysorbate 20 was employed as partial substitution of HP-CD to solubilize the essential oil in water due to relatively low cost. Incorporation of the surfactant to the formulation was found to improve water solubility of the essential oil when less amount of HP-CD was used. Formulation 2 demonstrated that addition of 3.24% (w/w) of the surfactant reduced the HP-CD content by 60% (w/w) to solubilize 0.825% (w/w) of the essential oil. A further reduction of 22% (w/w) HP-CD content was observed when the content of polysorbate 20 was increased to 3.96%, giving a clear solution as observed in formulation 3.

2. Vaporization of Formulations at Different Temperatures:

The working temperature of the formulations was determined by studying the vaporization of the APIs at different temperatures. This was achieved by collecting the vapors generated from the formulations heated by the inhaler device, while a 1.2 L/min pump was used to direct the vapors out of the inhaler to the air sampling bag for collection. The vaporized amount of α-pinene, limonene and eucalyptol were studied by dissolving the collected vapors in hexane and analyzed by GC-FID.

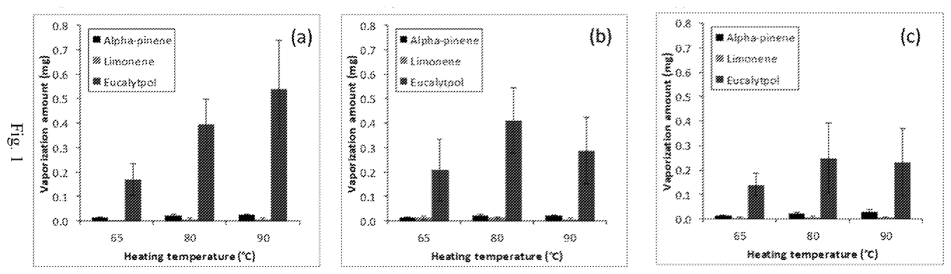

Formulations 1, 2 and 3 were each tested by the inhaler device at 65° C., 80° C. and 90° C. The three formulations were composed of the same amount of Eucalyptus Globulus oil (i.e. 0.825% w/w), but in different polysorbate 20-to-HP-CD ratios of 0, 0.9 and 2.4, respectively. The vaporized amount of α-pinene, limonene and eucalyptol from the formulations was presented in FIG. 1. At 65° C., formulations 1, 2 and 3 released ˜0.013-0.015 mg of α-pinene in the vapors. Increasing the heating temperature to 80° C. and 90° C. did not increase the concentration of the API significantly. Formulations 1 and 3 exhibited a similar trend of limonene release of 0.002-0.007 mg at the three heating temperatures. In comparison with the two formulations, formulation 2 showed a six-fold and two-fold increase in limonene vapors released at 65° C. and 80° C., respectively. However, the amount of limonene vapors at 90° C. remained similar to formulations 1 and 3 at the same temperature. Eucalyptol was released in relatively large amount in the vapors among the three APIs as it was the major component in Eucalyptus Globulus oil. Eucalyptol amount in vapors increased from 0.17 mg to 0.54 mg when formulation 1 was heated from 65° C. to 90° C. Eucalyptol vapors from formulation 2 remained at 0.2-0.3 mg at 65° C. and 90° C. The amount increased to 0.4 mg when the formulation was heated at 80° C. Formulation 3 showed a 0.14 mg release of eucalyptol vapors at 65° C. Increasing the heating temperature to 80° C. and 90° C. increased the eucalyptol amount to 0.23-0.25 mg.

Although the formulations released a relatively high amount of APIs at 90° C., a heating temperature of 65° C. was chosen for further examples since comparable amount of APIs could be released under this condition. Furthermore, the working temperature below 75° C., preferably 65° C. could reduce the risk of burn in the upper respiratory area during inhalation of the vaporized formulation. In particular, comparable amount of APIs of formulation 2 could be released at 65° C.

3. Oil Loading of Formulations:

Formulation was studied based to the relationship between maximum loading of Eucalyptus Globulus oil and polysorbate 20-to-HP-CD weight ratio. The analysis was performed by introducing maximum possible amount of the essential oil to the formulations in different polysorbate 20-to-HP-CD ratios without causing turbidity of the solution after stirring. The water content in the formulations was kept from 60% to 45% at polysorbate 20-to-HP-CD ratio of 0-11.4 to ensure complete dissolution of HP-CD in water.

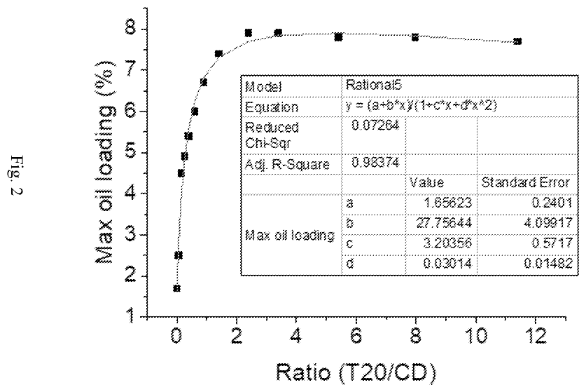

The relationship between maximum essential oil loading andpolysorbate 20-to-HP-CD ratio was summarized in FIG. 2. Oil loading increased to about 8% at polysorbate 20-to-HP-CD ratio of 2.4. The maximum oil loading remained at ˜8% when polysorbate 20-to-HP-CD ratio was increased to 11.4. Equation A was derived from the fitted curve to determine the maximum oil loading in the formulation at each polysorbate 20-to-HP-CD ratio, where the term “CR” represents the polysorbate 20-to-HP-CD.

$$Max oil loading ( \% ) = \frac{1.656 + 27.756 \times CR}{1 + ( 3.204 \times CR ) + ( 0.090 \times {CR}^{2} )} \qquad Equation A$$

Another example of formulation was based on formulation 2 in polysorbate 20-to-HP-CD ratio of 0.9. Substituting CR with 0.9 in Equation A would give a maximum oil loading of 6.8%, giving formulation 4. For a daily dosage of Eucalyptus Globulus oil of 35 mg by inhalation, formulation 4 would supply 68 mg of the essential oil assuming 1 g of the formulation was used for each dosage. Aiming the keep the essential oil supply within a safe inhalation limit, formulation 4 was diluted by three-fold, named as formulation 9. The essential oil concentration in this formulation 9 was 23 mg/g, which was within the recommended daily dosage.

4. Determining the droplet size of vapors from the formulation:

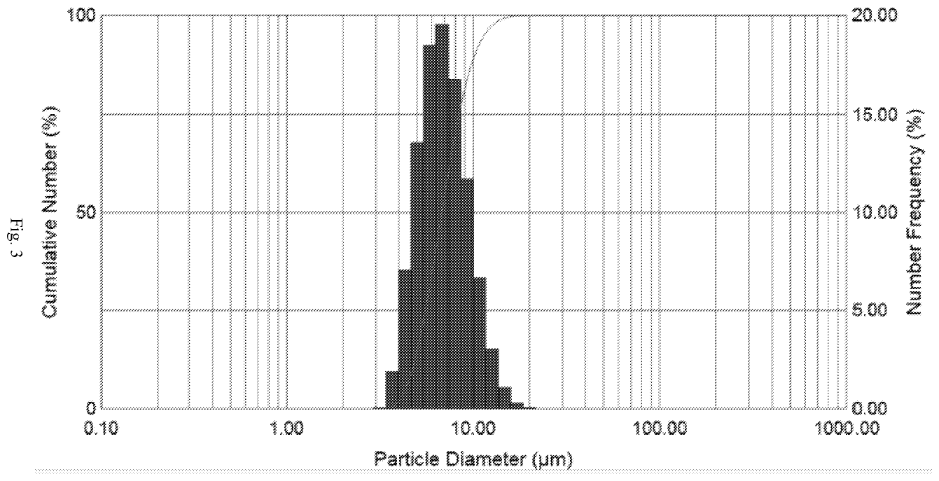

The size of vapor droplets generated from formulation 9 at 65° C. was measured by laser diffraction system. The sample was heated in a petri dish for measurement. This ensured sufficient vapors was generated from the sample surface for detection. FIG. 3 shows that the measured vapors exhibited a unimodal particle, with a mean Dn(50) particle diameter of 7.20±0.36 μm (Table 4).

TABLE 4
Droplet size of vapors from formulation 5 at 65° C. (n = 3)

| | Dn(10)¹ | Dn(50)² | Dn(90)³ | Span⁴ | Average Dn(50) | SD |
|---|---|---|---|---|---|---|
| Run | [μm] | [μm] | [μm] | [μm] | [μm] | [μm] |
| 1 | 4.7 | 6.8 | 10.2 | 0.8107 | 7.20 | 0.36 |
| 2 | 5.2 | 7.3 | 10.7 | 0.7581 | (n = 3) | (n = 3) |
| 3 | 5.3 | 7.5 | 10.9 | 0.7444 | | |

¹Dn(10) = the diameter of particles separating the lower 10% of a distribution
²Dn(50) = the diameter of particles separating the lower 50% of a distribution
³Dn(90) = the diameter of particles separating the lower 90% of a distribution
⁴Span = [Dn(90) − Dn(10)]/Dn(50)

5. In Vitro Evaluation of the Aerodynamic Droplet Size of Vapors from the Formulation:

The aerodynamic droplet size of vapors from formulation 5 was analyzed in vitro through Next Generation Impactor (NGI). Vapors generated from the formulation during heating at 65° C. were pumped into the NGI at a flow rate of 37 L/min for 10 minutes. Vaporized content from the formulation was deposited into the NGI stages corresponding to different cut-off sizes at the tested flow rate.

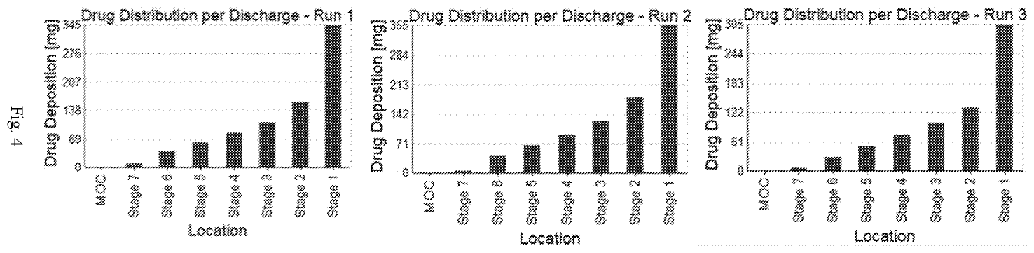

The weight distribution of the vaporized content deposited in the collection stages is summarized in FIG. 4. Stage 1 showed the largest amount of deposited content from the vapors, followed by stages 2-7. The distribution of the deposited vapors gave a mean MMAD of 8.48±0.26 with over 70% of the content was larger than 4 μm (Table 5). The measured aerodynamic droplet size correlated with the droplet size of vapors measured by laser diffraction system.

TABLE 5
Particle size distribution of vaporized formulation
5 analyzed by NGI/CITDAS (n = 3).

| | | | Vapors > | Mean vapors > |
|---|---|---|---|---|
| | MMAD⁵ | Mean MMAD | 4 μm | 4 μm |
| Run | [μm] | [μm] | [%] | [%] |
| 1 | 8.56 | 8.48 ± 0.26 | 73.80 | 74.0 ± 0.3 |
| 2 | 8.20 | (n = 3) | 73.80 | (n = 3) |
| 3 | 8.70 | | 74.40 | |

⁵MMAD = Mass median aerodynamic diameter, which is defined as the diameter at which 50% of the particles by mass are larger and 50% are smaller.

6. Screening for Harmful Chemicals in Formulation Generated During Heating:

Formulation 4 was chosen for the screening of harmful chemicals since it was composed of the highest amount of oil content among other formulations. The formulation was refluxed at 100° C. for an hour to ensure the volatile chemicals were generated for characterization. A total of 59 listed organic chemicals (belonged to Class 1 to Class 3) were screened in accordance with USP Chapter <467>. The test report stated that none of the organic chemicals had exceeded the corresponding upper limits recommended by USP.

7. Evaluation of API Content in Vapors from Formulation During 30-minute Heating

Vaporization profile of the APIs released from formulation 5 during heating was studied. This was achieved by heating the formulation at 65° C. continuously with the inhaler for 30 minutes, while vapor samples were collected at 5-minute intervals of the heating and analyzed by GC-MS. The experimental set-up mimicked the normal human breathing condition by employing a 6 L/min pump to direct the vapors out of the inhaler, while approximately 0.4-0.5 L vaporized sample was collected at each time point. The selected pump flow rate resembled the human minute ventilation (defined by the total amount of air moved in and out of the respiratory system each minute) at rest. The collected amount of vaporized sample at each time point was equivalent to the volume of air inhaled in each breath (also known as tidal volume) by a normal human at rest.

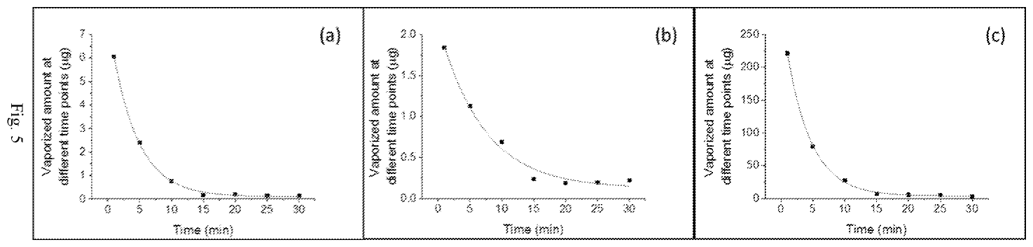

The vaporization profile of the formulation is summarized in FIG. 5. The amount of released APIs was found to decrease exponentially between 1 minute and 30 minutes of heating. Relatively high level of eucalyptol was detected in the vapors as the API was the major component in the essential oil. On the hand, limonene content was found to be the lowest among the three APIs in the vapors. Apart from its low abundance in the essential oil, this is due to its relatively low vapor pressure compared to other APIs at fixed temperature. Overall, the vaporization trend showed that the optimal time for inhaling 1 mL of the formulation was within 15 minutes, since most of the APIs were released during the time frame.

### Example 9—In Vivo Studies of Formulation

This example explores the in vivo performance of formulation 5. Animal model was allowed to inhale the vapors released from the formulation heated at 65° C. Deposition and bioavailability of the APIs and their corresponding metabolites in rat organs, in particular, the upper respiratory system, and tissues were compared. The deposition efficiency of the APIs in the animal model in the in vivo tests was also determined.

In vivo tests were performed on 6 to 8-week old male Sprague Dawley rats (Lo Kwee-Seong Integrated Biomedical Sciences Building, Chinese University of Hong Kong), each weighing 200-220 g. The rats were anaesthetized by a mixture of ketamine (75 mg/kg)/xylazine (10 mg/kg) prior to administration of the formulation.

1. Vapor Droplet Deposition in Animal Model

Briefly, 1 mL of formulation 5 was fed into the inhaler device and heated at 65° C. for a fixed time. Twenty anaesthetized rats were divided into four groups and allowed to inhale the vapors from the heated formulation for either 1, 5, 10 or 15 minutes (n=5 for each time point). Another four rats served as the negative control by inhaling ultrapure water instead of the formulation at each time point under the same testing condition (n=1 for each time point). Vapors from the formulation/water were delivered to the low profile anesthesia mask (Model VetFlo-0803, Kent Scientific Corporation) by pumping the outlet of the inhaler device at a flow rate of 1.5 L/min. After each inhalation time point, the rats were sacrificed immediately by an overdose of ketamine-xylazine cocktail. Larynxes, tracheas, and lungs were harvested and stored at −80° C.

2. Extraction of APIs from Organs and Tissues

2.1 Larynx and Trachea

Frozen larynx and trachea were thawed at room temperature and their weights were recorded. The whole larynx was used for extraction of API and metabolites, while trachea was cut into five pieces for the extraction. The (cut) organs were vortexed in 3 mL hexane for 1 minute and the upper layers were transferred into 10-mL volumetric flasks. The extraction was repeated two more times for both organs. Supernatants were combined and marked up to 10 mL with hexane, following by mixing. Samples were filtered through 0.45 μm PTFE filters and analyzed by GC-MS.

2.2 Lung

Frozen lungs were defrosted at room temperature. The weights of left and right lungs were recorded separately. About 0.3 g of left lung was obtained and cut into small pieces for extraction. The cut organ was blended with 2 mL water by ultrasonication (Ultrasonic probe sonicator 500 Watt, Ultra Autosonic) at 100% amplitude in an ice bath for 3 minutes. Blended organ was then vortexed in 3 mL hexane for 1 minute, followed by centrifugation at 7000 rcf for 8 minutes. The supernatant was transferred into a 10-mL volumetric flask. The extraction was repeated two more times. Supernatants were combined and marked up with hexane, followed by mixing. Samples were filtered through 0.45 μm PTFE filters and analyzed by GC-MS.

3. Result

3.1 Vapor Droplet Deposition in Animal Model

In vivo vapor droplet deposition performance of formulation 5 was evaluated. This was achieved by heating 1 mL of the formulation at 65° C. with the inhaler device to generate vapors for the rats. The rats were sacrificed immediately after inhaling the formulation for 1, 5, 10 and 15 minutes. Organs from the respiratory system were collected for analysis of API content.

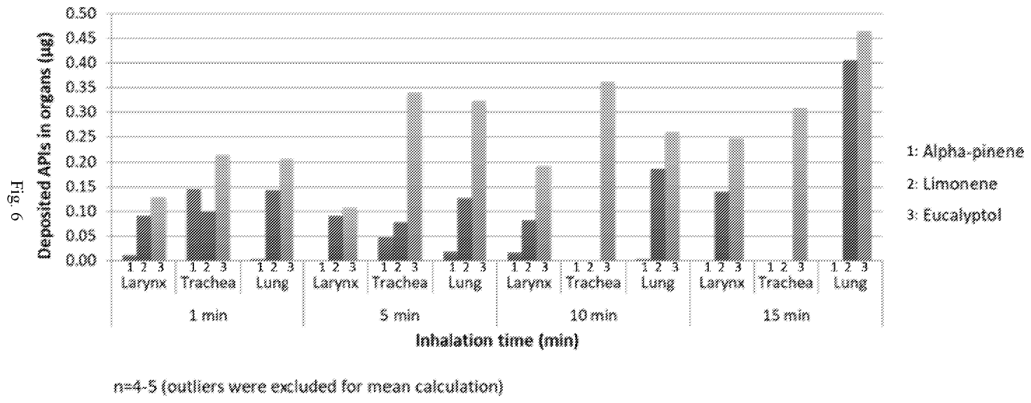

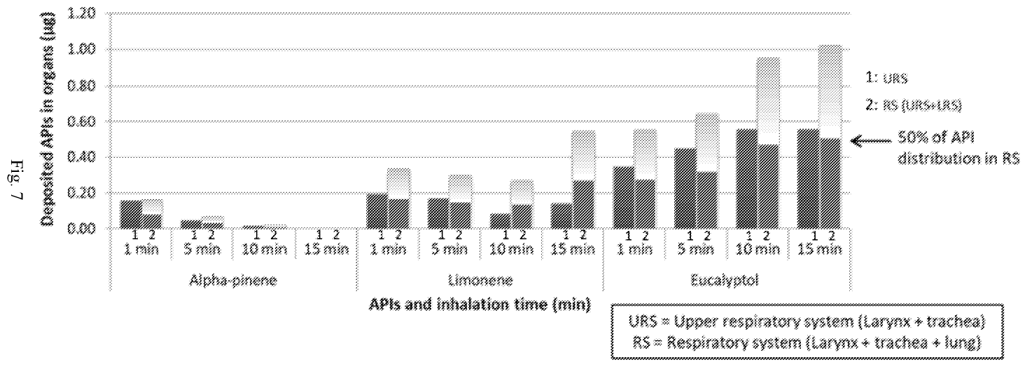

The distribution profile of the APIs in the respiratory system is summarized in FIG. 6. In general, limonene and eucalyptol deposition increased with inhalation time. The deposition of α-pinene was found to decrease from 1-minute to 10-minute inhalation. No α-pinene deposits were found at 15-minute inhalation. The total amount of APIs deposited in the upper respiratory area (URS) against the whole respiratory system (RS) was evaluated. URS was represented by larynx and trachea, while RS included the larynx, trachea and lung. FIG. 7 compares the distribution of the three APIs between the URS and the RS. More than 50% of the three APIs were deposited in the URS when the formulation was inhaled for 1 minute and 5 minutes. At 10- and 15-minute inhalation time, less than 50% of limonene was deposited in the URS.

1. A composition, comprising: hydroxypropyl-β-cyclodextrin (HP-CD), an essential oil, water, optionally a surfactant and optionally a stabilizer,
wherein the essential of is selected from a group consisting of Eucalyptus Globulus oil and Houttuynia cordata oil,
wherein the surfactant is polysorbate, and
wherein the stabilizer is polyhydroxy alcohol.

2. The composition of claim 1, wherein the composition comprises 1.6%-28% HP-CD, 3%-25% polysorbate 20, 0.8%-7% Eucalyptus Globulus oil, and 41%-94% water in weight % of the composition.

3-7. (canceled)

8. The composition of claim 1, wherein the composition consists essentially of about 14.8% HP-CD, about 0.68% Eucalyptus Globulus oil, about 79.52% water, and about 5% propylene glycol.

9. The composition of claim 1, wherein the composition consists essentially of about 11% HP-CD, about 0.18% Houttuynia cordata oil and about 88.82% water.

10. (canceled)

11. The composition of claim 1, wherein when the composition is at a temperature at a range of 65-75° C., vapors of the composition have a droplet size of 1-10 μm.

12. The composition of claim 1, wherein when the composition is at a range of 65-75° C., vapors of the composition have a droplet size of 4-10 μm.

13-14. (canceled)

15. The composition of claim 1, wherein the HP-CD encapsulates the essential oil in hydrophobic cavities of the HP-CD so that the essential oil is solubilized in the water.

16. The composition of claim 1, wherein the polysorbate is polysorbate with a hydrophile-lipophile balance of 14.9-16.7.

17. The composition of claim 1, wherein the composition comprises 0.3%-0.7% Houttuynia cordata oil, 5%-11% polysorbate 20, 5%-11% HP-CD, and 77%-89% water in weight % of the composition.

18-19. (canceled)

20. A method of targeting an inflammation disease of the respiratory tract in a subject in need thereof, comprising: administering a composition of claim 1 contained in an inhaler to the subject.

21. The method of claim 20, wherein the respiratory tract is the upper respiratory tract.

22. The method of claim 20, wherein the inhaler includes a heater that heats the composition to form vapors with a droplet size of 1-10 μm.

23. The method of claim 20, wherein the inhaler includes a heater that heats the composition to form vapors with a droplet size of 4-10 μm.

24. The method of claim 23, wherein vapors of the composition are inhaled by the subject for 1-10 minutes to target the inflammation disease of the upper respiratory tract.

25. (canceled)

26. An inhaler, comprising: a pod that includes a composition of claim 1; and a heater configured to heat the composition to a temperature at a range of 65-75° C.

27-28. (canceled)

29. A method of making a composition of claim 1, comprising:
mixing HP-CD into water to obtain a HP-CD solution; and
adding the essential oil and optionally the surfactant/optionally the stabilizer to the HP-CD solution at room temperature to obtain a mixture.

30. The method of claim 29, wherein the mixing HP-CD into water further includes:
mixing HP-CD with a weight of 1.6%-28% of the composition into water with a weight of 41%-94% of the composition to obtain the HP-CD solution;
wherein the adding the essential oil further includes:
adding Eucalyptus Globulus oil with a weight of 0.8%-7% of the composition, and polysorbate 20 with a weight of 3%-25% into the HP-CD solution with constant stirring at room temperature to obtain the mixture;
vortexing the mixture for 1 minute and degassing the mixture for 30 seconds to obtain the composition.

31. (canceled)

32. The method of claim 29, wherein the mixing the HP-CD into the water further includes:
mixing HP-CD with a weight of 14.8% of the composition into water with a weight of 79.52% of the composition to obtain the HP-CD solution;
stirring the HP-CD solution for 15 minutes at 600 rpm; and
filtering the HP-CD solution by 0.2μm membrane to obtain a filtrate, and wherein the adding the Eucalyptus Globulus oil further includes:
adding Eucalyptus Globulus oil with a weight of 0.68% of the composition to the filtrate and stirring for 18 hours at 600 rpm to obtain a solution; and
adding propylene glycol with a weight of 5% of the composition into the solution and stirring at 600 rpm for 10 minutes to obtain the composition.

33. The method of claim 29, wherein the mixing HP-CD into water further includes:
mixing HP-CD with a weight of 11% of the composition into water with a weight of 88.82% of the composition to obtain the HP-CD solution;
stirring the HP-CD solution for 15 minutes at 600 rpm; and
filtering the HP-CD solution by 0.2μm membrane to obtain a filtrate, and wherein the adding Houttuynia cordata oil further includes:
adding Houttuynia cordata oil with a weight of 0.18% of the composition to the filtrate and stirring for 18 hours at 600 rpm to obtain the composition.

34. The method of claim 29, wherein the mixing HP-CD into water further includes:
mixing HP-CD with a weight of 5%-11% of the composition into water with a weight of 77%-89% of the composition to obtain the HP-CD solution;
votexting the HP-CD solution for 1 minute;
and wherein the adding Houttuynia cordata oil further includes:
adding Houttuynia cordata oil with a weight of 0.3%-0.7% of the composition to polysorbate 20 with a weight of 5%-11% by stirring for 5 minutes to obtain an oil solution; and
mixing the HP-CD solution with the oil solution by vortexing for 1 minute and degassing for 30 seconds to obtain the composition.

35. (canceled)
